(19) United States
(12) Patent Application Publication
(10) Pub. No.: US 2007/0225572 A1
(43) Pub. Date: Sep. 27, 2007

(54) System & an apparatus for inspection of feet

(76) Inventor: Albert Murillo, Templeton, CA (US)

Correspondence Address: LAW OFFICES OF ANDREW Y. SCHROEDER, 500 SOUTH BROADWAY, SUITE 222, SANTA MARIA, CA, 93454, US

(21) Appl. No.: 11/348,920

(22) Filed: Feb. 6, 2006

(51) Int. Cl. A61B 1/00

(52) U.S. Cl. 600/247

(57) ABSTRACT

A structure for use at home or in a doctor's office to support an assembly of mirrors such that sides of the structure facing the seated patient are transparent or open and enable a patient and/or doctor/health caregiver to simultaneously completely examine all sides of a patient's foot including a top side, a bottom side, lateral sides, and heel of the foot.

### CROSS-REFERENCE TO RELATED APPLICATIONS

System & Apparatus for Inspection of Feet application Ser. No. 11/136,352

### FIELD OF THE INVENTION

The present invention is in the area of medical equipment, and more specifically relates to apparatus and methods for inspecting feet.

### BACKGROUND OF THE INVENTION

Diabetes poses grave health dangers to people which include death, amputation, and gangrene. If people suffering with diabetes do not properly regulate their diet, check for gangrene and the other safety protocols associated therewith, serious consequences may ensue.

One particular problem Diabetes poses relates to the feet of the victims. Oftentimes, complications such as blisters, bruises, rashes, in-grown toenails, etc on a diabetes victim will manifest themselves on the soles of a victim's foot. If these problems are not properly treated in time, these complications will exacerbate to the point where the foot becomes infected with gangrene. Once gangrene sets in and accumulates in one's foot, a doctor may have no choice to amputate the gangrenous infected area in order to prevent its spread to the rest of the body.

Unfortunately, many Diabetes patients are elderly people who cannot properly examine the soles of their own feet (Diabetes can also affect the young and sometimes manifest with babies). These elderly people have less flexibility and are oftentimes overweight. Therefore, the only way to ensure proper inspection of their own feet is to either have a doctor do it for them or have another person inspect it. However, having other people inspect one's own feet may be awkward and a bit embarrassing.

Currently, the marketplace does not provide for a method or apparatus for the self-inspection of one's own feet. Therefore, what is clearly needed is an apparatus, system and methods thereof to properly inspect one's own feet daily.

### SUMMARY OF THE INVENTION

A structure for use at home, at work, on vacation, or in a doctor's office to support an assembly of mirrors such that sides of the structure facing the seated patient are transparent or open and enable a patient and/or doctor/health caregiver to completely examine all sides of a patient's foot including a top side, a bottom side, lateral sides, and heel of the foot.

### BRIEF DESCRIPTION OF THE DRAWING FIGURES

FIG. 1a is a perspective view of an exemplary system according to an embodiment of the present invention.

FIG. 1b is a perspective view of an exemplary system according to a preferred embodiment of the present invention.

FIG. 1c is a plan view of a preferred embodiment of the present invention.

FIG. 1d is a perspective view of a preferred embodiment of the present invention.

FIG. 1e is a perspective view of a preferred embodiment of the present invention.

FIG. 1f is a perspective view of a preferred embodiment of the present invention.

FIG. 1g is a perspective view of a preferred embodiment of the present invention.

FIG. 2a is a is a perspective view of an exemplary embodiment of the present invention.

FIG. 2b is a perspective view of a preferred embodiment of the present invention.

FIG. 2c is a perspective view of a preferred embodiment of the present invention.

### DESCRIPTION OF PREFERRED EMBODIMENTS

According to a preferred embodiment of the present invention, a unique method, system, and apparatus is used to inspect the feet of a person. This foot inspection may be used especially by diabetes patients to search for blisters, bruises, rashes, in-grown toenails, red spots, etc. on the soles of their feet. Moreover, this system is designed to be used by health caregiver such as medical doctors, podiatrists, nurses, physician's assistants, etc. for the purpose of evaluating and diagnosing any feet related maladies. In some preferred embodiments, the present invention is used by a health caregiver to show the patient where there are any feet related maladies and use the present invention to explain to the patient how both the health caregiver (e.g. A doctor) or the patient can treat said maladies. The present invention is also designed to be used at home, at the office, on vacation, or at any other place by people for the purpose of self-evaluation. The method, apparatus, and system may also be used for other foot inspection purposes as well. The method, apparatus, and system are described in enabling detail below.

The present invention is designed such that a patient may evaluate a person's foot through the view given by the top mirror 108. The present invention also enables a health caregiver to use the lower mirrors in such a way to examine all regions of the feet from top to bottom as well as the sides of the feet. The present invention will also enable both the patient and/or health caregiver simultaneously examine the foot from the same angle and in the same light.

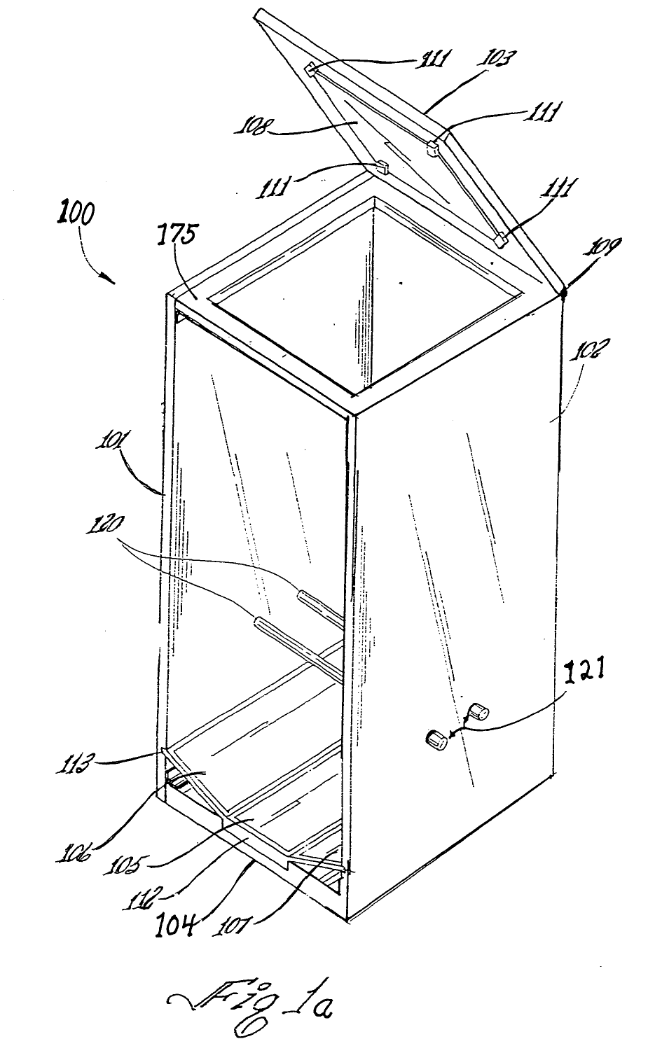

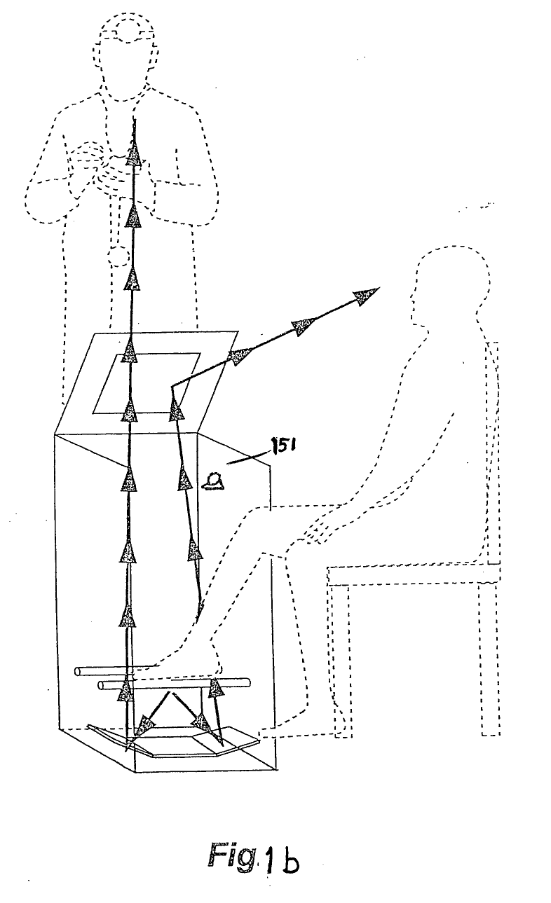

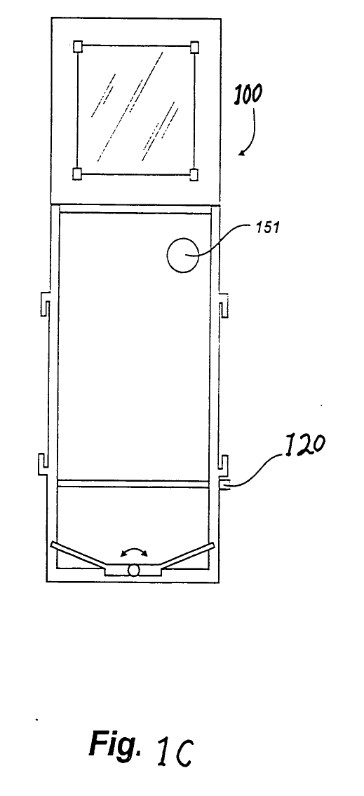

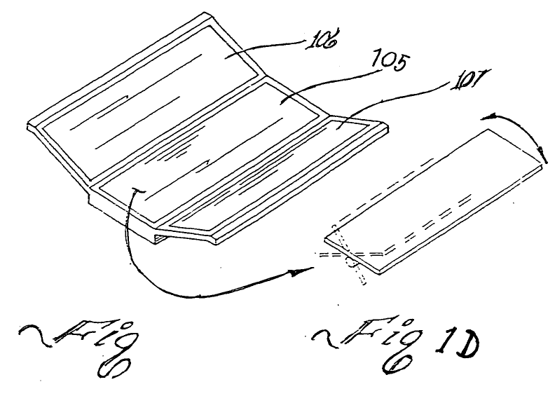

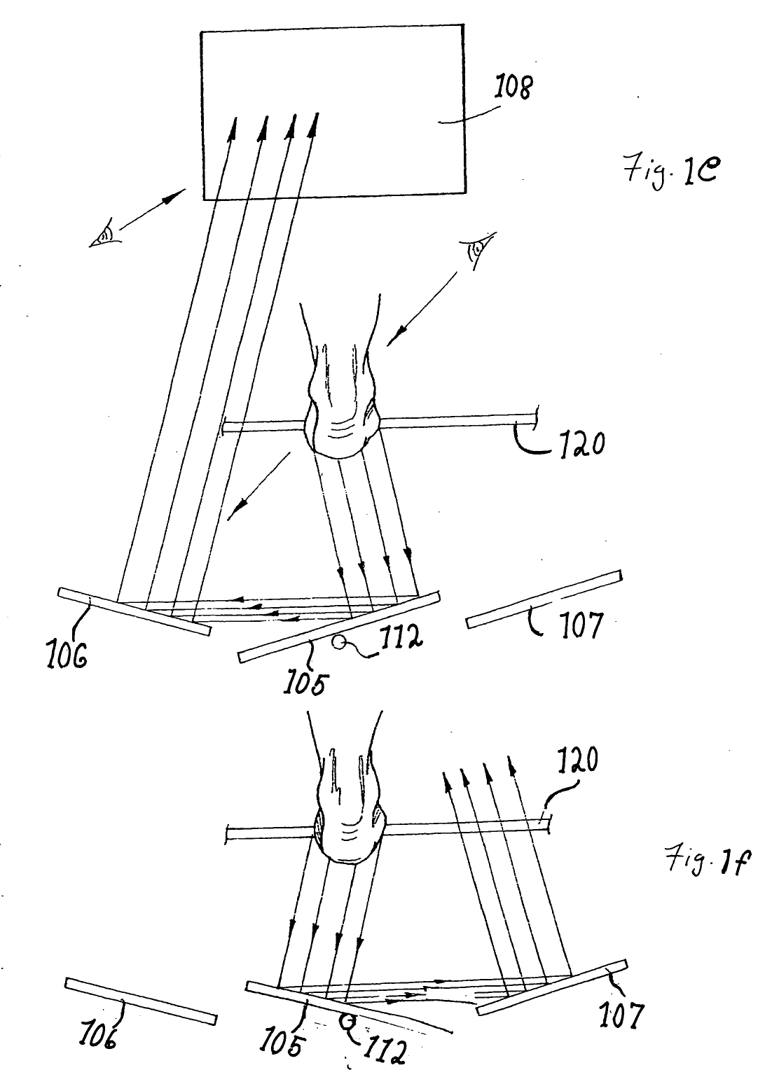

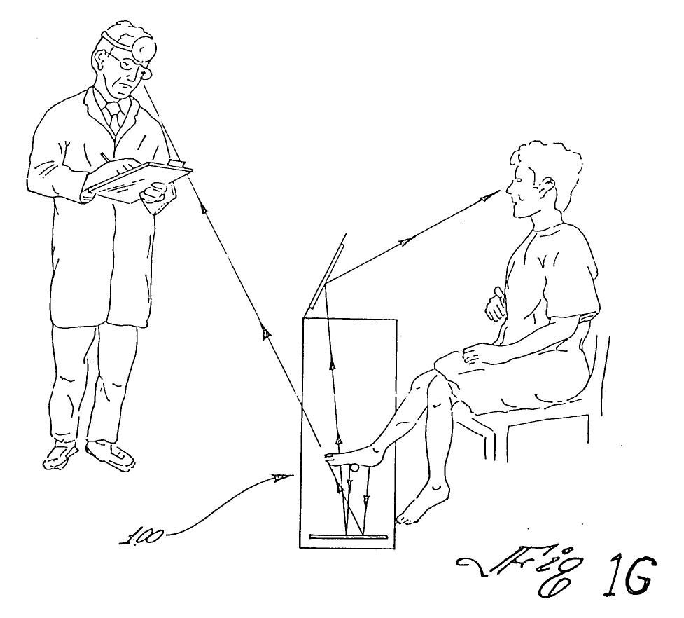

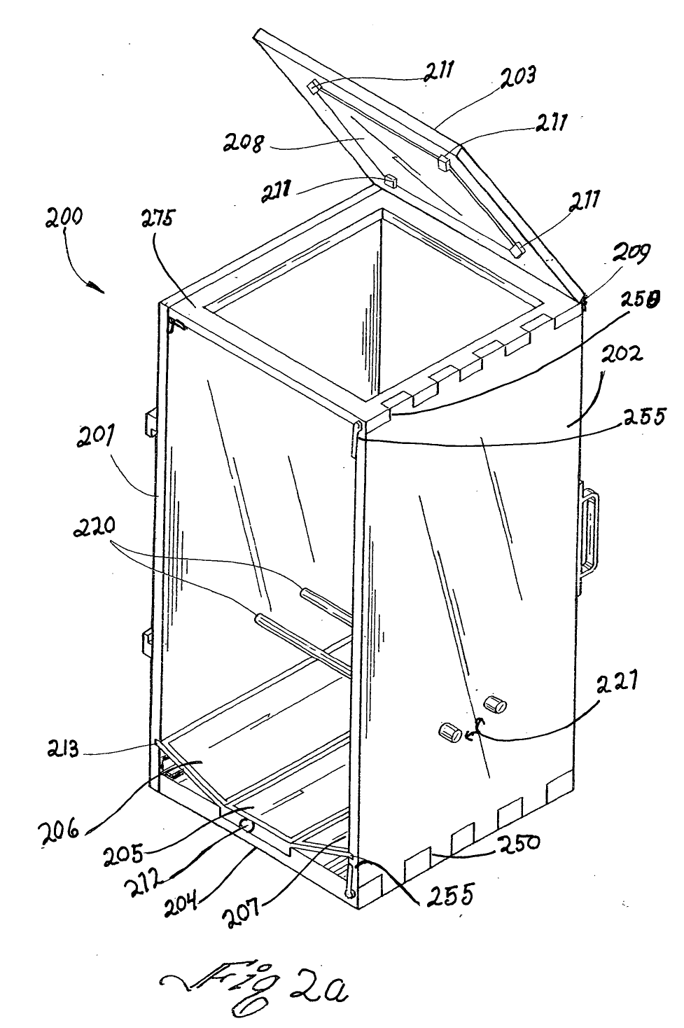

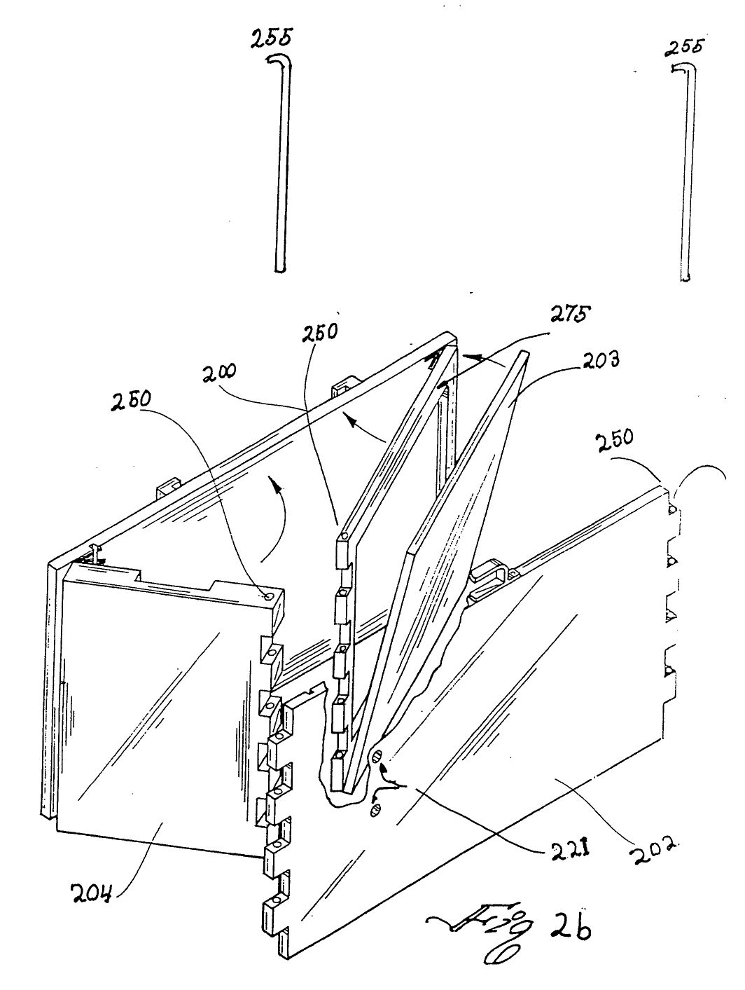

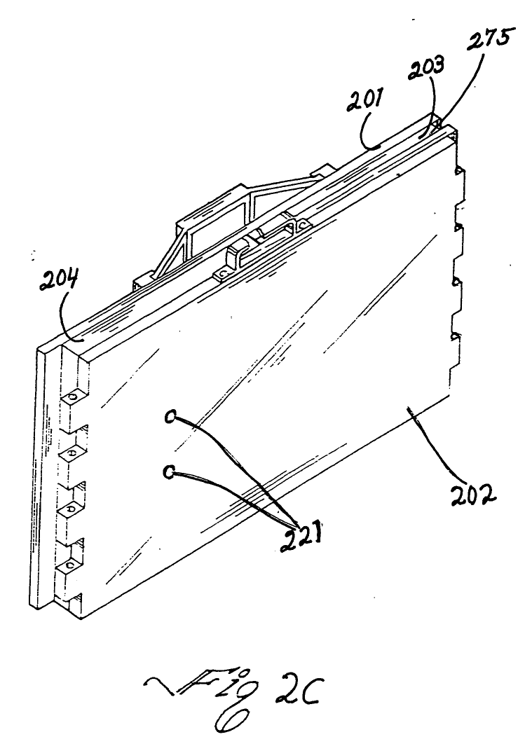

It will be pointed out here that there are two main preferred embodiments of the present invention. FIG. 1a illustrates a stationary preferred embodiment. FIGS. 2a-2c illustrates a portable preferred embodiment of the present invention. The preferred embodiment illustrated in FIGS. 2a-2c is collapsible such that it may be easily stored and easily transported. The preferred embodiment illustrated in FIG. 2a-2c shall be referred to hereafter as the “portable embodiment”.

FIG. 1a shows a preferred embodiment of the present invention. The vertical wall 101 is attached with screws to the left bottom side of bottom horizontal wall 104. Left, top of vertical wall 101 is attached by screws to the left side of top horizontal frame 175. The right vertical wall 102 is attached with screws to the right top side of the horizontal frame 175. The bottom side of the right vertical was 102 is attached with screws to the right bottom side of the horizontal wall 104. Only the top lid holding the mirror is hinged to the front side of the horizontal frame 175. The stationary model has only one hinge so the sitting patient may view his or her feet. Both models (the stationary and the collapsible) have the same top mirror design, hinge to front, side of horizontal frame 175.

Apparatus frame 100 comprises vertical wall 1 101, vertical wall 2 102, top lid 103, top frame 175 and bottom wall 104. In addition, a light source 151 may also be used in some preferred embodiments. FIG. 1c shows a preferred embodiment where light source 151 may be affixed to any of the walls. Attached to bottom wall is bottom mirror 105, side mirror 1 106, and side mirror 2 107. Top mirror 108 is affixed to top lid 103 by affixing means 111. FIGS. 1d, 1e, and 1f are other views of the bottom mirrors.

FIGS. 1b, 1e, 1f and 1g illustrate the light paths by which a patient and doctor will see the feet of the patient. Light paths may be altered by tilting the mirrors on the bottom.

Apparatus frame 100 may be composed of a panoply of materials. Just about any type of material may be used in order to make the apparatus operationally functional. Exemplary materials include plastic, wood, polymer, steel, aluminum, cardboard, and even fabric. In a preferred embodiment, the composition of apparatus frame 100 is lightweight.

Bottom mirror 105 in a preferred embodiment is magnified for the purpose of proper inspection of feet. Bottom mirror 105 in some embodiments may have a regular mirror which is not magnified on the opposite side. Therefore, bottom mirror 105 in some embodiments is double-sided: a magnified mirror on one side and a regular mirror on the other side. Moreover, bottom mirror 105 is independently inclined about an axis which is parallel with the base plane of the support structure and perpendicular to the two transparent sides of the support structure thereby enabling a viewing position through the three bottom mirrors for an attendant health caregiver who is facing the seated patient.

Side mirror 1 106 and side mirror 2 107 are angled in such a fashion such that they face each other. All three mirrors are affixed to Apparatus frame 100 via affixing means 111. Affixing means 111 are inconsequential to the present invention because there are a panoply of means to do so. A person skilled in the art would be able to quickly discern how to do so. Moreover, as with the bottom mirror 105 is inclinable about an axis as is illustrated by FIG. 1d. Side mirrors 106 and 107 are stationary and do not move in this particular embodiment. This adjustability enables viewing of the foot by either the patient individually, or by the health caregiver. The adjustability also enables one to see different angles and all regions of a bottom of one's foot.

FIG. 1a illustrate that affixed substantially in the lower portion of the Apparatus frame 100, 200 are feet holders 120 or foot rest. In a preferred embodiment feet holders 120 are long elongated rods which are affixed to Apparatus frame 100, 200 by simply placing feet holders 120 through their respective holes 121 in Apparatus frame 100 200, through one side and out the other. It is preferred that the rods which comprise the feet holders 120 are of a minimum cross-sectional diameter in order to minimize any obstruction of the view of the underside of one's foot.

The composition used for feet holders 120 is of no consequence. Any material which is strong enough to withhold the weight of a person's feet/leg upon the feet holders 120 is expedient for the task. In preferred embodiments feet holders 120 are made of steel, plastic, polymer, or even aluminum.

FIG. 2a illustrate another preferred embodiment of the present invention which is made to be collapsible. Portable embodiment 200 comprises vertical wall 1 201, vertical wall 2 202, top lid 203, top frame 275 and bottom wall 204. Vertical wall 2 202 has a dovetail shape to fit and conjoin with top frame 175 and the right side of bottom wall 204. A hole 250 is located longitudinally through the dovetail edges to house locking member 255. Locking member 255 is made to fit into hole 250 in order to lock vertical walls with top wall 275. FIGS. 2b and 2c illustrates how the portable embodiment resembles a suitcase for easy transporting and storage.

FIG. 2a illustrates a preferred embodiment of the present invention and shows how a preferred embodiment of the present invention may be collapsed in order to become portable. First, the feet holders 220 or foot rest are removed from their respective holes 221. The top frame 275 is then disconnected from vertical wall 202 by pulling out locking member 255 from the hole 250. Side wall 201, in this embodiment is attached to bottom 204 and top frame 275 by hinges. And both top frame 275 and bottom wall 204 is connected with vertical wall 2 202 through cooperating dovetail shapes. Then, top frame 275 is placed against vertical wall and the other vertical wall is disconnected. The same process is also performed for the other movable portions of the present invention. The present invention is not limited to this particular mode of assembly and disassembly.

What is claimed is:

1. A structure for use at home or in a doctor's office to support an assembly of mirrors such that sides of the structure facing the seated patient are transparent or open and enable a patient and/or doctor/health caregiver to completely examine all sides of a patient's foot including a top side, a bottom side, lateral sides, and heel of the foot.

2. The structure of claim 1 further comprising feet holders located above the first mirror and substantially parallel with the base of the structure.

3. The structure of claim 2 wherein the feet holders consist of two parallel bars with minimum cross sectional area that might obscure viewing all areas of the foot.

4. The structure of claim 3 wherein said structure includes at least two mirrors, one of said mirrors is located below a foot rest and the other mirror located above the foot rest and adjustably inclined with respect to the first mirror such that a seated patient can look straight forward into the second mirror and view the reflected image of his foot from the first mirror while his foot in a relaxed position on the foot rest.

5. The structure of claim 2 further comprising two angled mirrors below the foot rest such that the patient can view the bottom and both sides of his foot while the foot is resting in a relaxed position in front of the patient.

6. The structure of claim 3 wherein the mirrors under the foot are each independently inclined about an axis which is parallel with the base plane of the support structure and perpendicular to the two transparent sides of the support structure thereby enabling a viewing position through only the bottom mirror for a doctor or a health caregiver who is facing the seated patient.

7. The structure of claim 6 wherein both a patient and a caregiver can view the foot together and discuss detailed conditions of the foot at the same time whereby the patient examines a foot through the top mirror and whereby the health caregiver examines a foot through the bottom mirrors.

8. The structure of claim 8 further including a focused light source mounted on the side perpendicular with the ground underneath the structure, said light source is aimed at the bottom mirrors such that the bottom of the foot is illuminated by reflection.

9. A structure to completely examine all sides of a patient's foot including a top side, a bottom side, lateral sides, and heel of the foot, said structure to support an assembly of mirrors such that sides of the structure facing the seated patient and the doctor or health caregiver for viewing are transparent or open, said structure comprising a foot rest, at least two mirrors, parallel sides, and a bottom support, said foot rest located above the assembly of mirrors and substantially parallel with the base of the structure, the foot rest can consist of two parallel bars with minimum cross sectional area that might obscure viewing all areas of the foot, said structure includes three mirrors located below the foot rest, one of said mirrors is located below the top lid mirror, and adjustably inclined with respect to the three bottom mirrors such that a seated patient can look straight forward into the top lid mirror and view the reflected image of his foot from the first mirror while his foot in a relaxed position on the foot rest.

10. An apparatus for inspecting feet comprising a left vertical wall, a right vertical wall, a top lid, a top frame, a bottom wall, a left side mirror, a right side mirror, a center mirror, and a top mirror;
said left vertical wall is attached to a first end of said bottom wall,
said right vertical wall is attached to a second end of said bottom wall,
said top lid is attached to top end of said left vertical wall and top end of said right vertical wall,
said top mirror is affixed to a bottom side of top lid via affixing means;
said center mirror is affixed substantially in the center to bottom wall;
said right side mirror is affixed to bottom wall substantially adjacent to said center mirror via affixing means;
said left side mirror is affixed to bottom wall substantially adjacent to said center mirror via affixing means.

11. The apparatus of claim 10 further comprising feet holding bars, said feet holding bars to pass longitudinally through holes located on said left vertical wall and said right vertical wall.

12. The apparatus of claim 10 wherein an upper end of left vertical member is hinged upon end of left side of top frame and an upper end of right vertical member is dovetailed shaped to engageably attach with said top frame, said top lid is dovetail shaped to engageably attach with said right vertical member and left vertical member.

13. The apparatus of claim 12 wherein said center mirror is magnified.

14. The apparatus of claim 12 wherein said center mirror is placed in a groove located on said bottom wall

15. The apparatus of claim 10 wherein said left side mirror is substantially tilted at an angle.

16. The apparatus of claim 10 wherein said right side mirror is substantially tilted at an angle.

17. The apparatus of claim 12 wherein said upper end of right vertical member and said lower end of right vertical member has a dovetail hole passing longitudinally through dovetail interfaces.

18. The apparatus of claim 12 further comprising locking bars which pass through said holes.
